(19) United States
(12) Patent Application Publication
(10) Pub. No.: US 2004/0219228 A1
(43) Pub. Date: Nov. 4, 2004

(54) Animal model for inflammatory bowel disease

(75) Inventors: Katharine Helen Banner, Sandwich (GB); Aleksandar Popovic, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/774,823

(22) Filed: Feb. 9, 2004

Related U.S. Application Data
(60) Provisional application No. 60/450,939, filed on Feb. 28, 2003.

(30) Foreign Application Priority Data
Feb. 11, 2003 (GB) 0303085.5

(51) Int. Cl. A01K 67/027; A61K 33/14

(52) U.S. Cl. 424/661; 800/18

(57) ABSTRACT

An animal model for inflammatory bowel disease is described. The model is based on a mammal not expressing a functional mdr1a gene product, or wherein the mdr1a gene product is inhibited, which develops inflammatory bowel disease when subjected to elevated chlorine concentrations, e.g. given chlorinated drinking water. Methods of screening for compounds useful for the treatment of inflammatory bowel disease, as well as methods to identify, for example, new targets for new compounds for the treatment of inflammatory bowel disease are also described.

### RELATED APPLICATIONS

This application claims benefit of priority under 35 U.S.C. § 119 to UK Provisional Application No. 0303085.5, filed Feb. 11, 2003, and under 35 U.S.C. § 119(e) to U.S. Provisional Application No. 60/450,939, filed Feb. 28, 2003, each of which is incorporated in its entirety by reference.

### FIELD OF THE INVENTION

The present invention relates to an animal model for inflammatory bowel disease. The model is based on a mammal not expressing a functional mdr1a gene product or wherein the mdr1a gene product is inhibited, which develops inflammatory bowel disease when subjected to elevated chlorine concentrations. More specifically, the mammal is a transgenic mouse lacking mdr1a.

This model facilitates the characterisation of the pathogenic mechanisms of inflammatory bowel disease and the development of diagnostics, therapies and therapeutic compounds.

### BACKGROUND TO THE INVENTION

Inflammatory bowel disease (IBD) is a chronic, relapsing and remitting inflammatory condition of unknown origin that afflicts individuals of both sexes throughout life. The disease is clinically characterised by two overlapping conditions, ulcerative colitis (UC) and Crohn's disease (CD). Clinical and laboratory studies in humans with IBD have suggested that genetic and environmental factors play an inter-related role in the pathogenesis of these disorders. More recent immunologic studies suggest that IBD may be due to a dysregulated mucosal immune response to one or more unknown antigens present in the normal bacterial gut flora.

The etiology of inflammatory bowel disease remains unknown despite many years of extensive investigation. Some insight into potential disease mechanisms has however been obtained from various animal models. For example, rodents deficient in genes encoding several cytokines including IL-2 (Sadlack B. et al (1993) Cell 75, 253-261), IL-10 (Kuhn R. et al. (1993) Cell 75, 263-274) and TGF-β (Shull M M. et al. (1992) Nature 359, 693-699) develop spontaneous intestinal inflammation. Selective gene deletion of proteins involved in T cell function, such as Gαi2 (Rudolph U et al (1995) Nat Genet 10, 143-150), TCRα (Mombaerts P. et al (1993) Cell 75, 274-282), and TCRβ also results in spontaneous intestinal inflammation. These studies have contributed to the hypothesis that the persistent inflammation seen in IBD may be the result of either enhanced or aberrant immunological responsiveness to normal constituents of the gut lumen, or an overall autoimmune dysregulation and imbalance.

Intestinal flora may also be an important co-factor in the pathogenesis of intestinal inflammation. For example, in several rodent models spontaneous colitis can be prevented if animals are maintained in a germ-free environment (Dianda L et al (1997) Am J Pathol 150, 91-97, Taurog J D et al (1994) J Exp Med 180, 2359-2364). Other studies have shown that intestinal inflammation can be successfully treated with oral antibiotics (Panwala C M et al. (1998) J Immunol 161, 5733-5744). In addition, patients with IBD have adverse and enhanced reactivity to their autologous resident flora (MacDonald T T. (1995) Clin Exp Immunol 102, 445-447). Further support for the hypothesis that intestinal flora may be important in the pathogenesis of intestinal inflammation has been derived from studies using mice with defects in epithelial barrier cell function, induced by deleting genes for trefoil factor (Mashimo H et al (1996) Science 274, 262-265) or N-cadherin (Hermiston M L & Gordon J I. (1995) Science 270, 1203-1207). These mice also have evidence of inflammation in the gastrointestinal tract. In addition to intestinal flora, it is plausible to hypothesize that food constituents may contribute to the inflammatory response observed in rodents with defective epithelial cell barriers, since they would potentially have access to areas of the gut lumen which they would not have in animals with an intact epithelial barrier.

Intestinal epithelial cells (Croop J M et al. (1989) Mol Cell Biol 9(3), 1346-1350; Gottessman M M & Pastan I. (1993) Annu Rev Biochem 62, 385427), and also some lymphocyte subsets (Bommhardt U et al. (1994) Eur J Immunol 24, 2974-2981) express multiple drug resistant (mdr) genes. Mdr genes encode P-glycoproteins, which are drug efflux pumps. These pumps are localised in the plasma membranes of many tissues and have a natural role to play in protecting the host from potentially harmful compounds. Three mdr1 genes have been identified in rodents (mdr1a, mdr1b and mdr2) and two in humans (mdr1 and mdr2). In mice, mdr1a P-glycoprotein is also expressed in the blood-brain and blood-testis barrier and mdr1b P-glycoprotein is expressed in adrenal glands, pregnant uterus and ovaries (Bommhardt U et al. (1994) Eur J Immunol 24, 2974-2981). Both mdr1a and mdr1b P-glycoprotein are expressed in the liver, kidney, spleen and heart.

In 1994, mice with a targeted deletion of the mdr1a gene were generated. These mice had an increased sensitivity to certain drugs, but did not appear to have any constitutive abnormalities (Schinkel A H et al (1994) Cell 77, 491-502). Mdr1a^{-/-} mice are routinely used in studies to evaluate the involvement of P-glycoprotein in the absorption of compounds, brain penetration of compounds and the hepatobiliary. excretion of compounds that are potentially substrates for P-glycoprotein. Interestingly, a recent study reported that approximately 25% of mdr1a^{-/-} mice spontaneously developed colonic inflammation that is histologically similar to human IBD (Panwala C M et al (1998) J Immunol 161, 5733-5744). We have observed similar clinical symptoms and lesions in mdr1a^{-/-} mice being used for drug metabolism studies, and preliminary data has been reported (Banner K H et al. (2001) Gastroenterology 120, A693). Specifically, clinical symptoms included acute body weight loss and soft “sticky” faeces and histopathological analysis revealed a marked diffuse colitis. However, with so few animals developing the symptoms of inflammatory bowel disease, this is not a reliable model that can be used on a routine basis for this disease.

In our work with this transgenic mouse strain for drug metabolism studies, we attempted to reduce the incidence of inflammatory bowel disease-like symptoms in this mouse strain by reducing the bacterial load in the drinking water through addition of chlorine. However, surprisingly, we found that addition of chlorine to the drinking water of the mdr1a^{-/-} mice led to a significant increase in the incidence of disease, as 100% of the animals which had been drinking chlorinated water developed inflammatory bowel disease-like symptoms which closely resembled the symptoms of inflammatory bowel disease in humans. This was highly unexpected, but it has led to the invention, the first reliable, reproducible animal model for inflammatory bowel disease which histologically closely resembles the human disease.

Now that a robust and reliable animal model is developed for this disease, which shows very similar characteristics to the disease as observed in patients suffering from inflammatory bowel disease, it becomes possible to use the model to study the etiology and progression of the disease and identify new targets for the development of compounds that will be beneficial for the treatment of the disease, be it to eliminate the cause of the disease or at least halt or slow the progression of the disease or treat the symptoms more effectively than currently available treatments.

### SUMMARY OF THE INVENTION

The present invention relates to an animal model for inflammatory bowel disease. The model is based on a mammal not expressing a functional mdr1a gene product, or wherein the mdr1a gene product is inhibited, which develops inflammatory bowel disease when subjected to elevated chlorine concentrations, e.g. given chlorinated drinking water. More specifically, the mammal is a transgenic mouse lacking mdr1a. Also included in the invention are methods of screening for compounds useful for the treatment of inflammatory bowel disease, as well as methods to identify, for example, new targets for new compounds for the treatment of inflammatory bowel disease.

### BRIEF DESCRIPTION OF THE FIGURES

FIG. 1: Effect of Dexamethasone on colon to body weight ratio in mdr1a^{-/-} mice (mean ±sem).

FIG. 2: Effect of Dexamethasone on caecum to body weight in mdr1a^{-/-} mice (mean ±sem).

FIG. 3: Height of the colon/rectum mucosa throughout the whole colon/rectum.

### DETAILED DESCRIPTION OF THE INVENTION

The invention is based on the surprising observation that a very high incidence of colitis (with symptoms and histology closely resembling human IBD) is observed in mdr1a^{-/-} mice when they are supplied with chlorinated drinking water. This observation has led to the development of a robust animal model for IBD, which is useful for testing compounds for their efficacy in treating, curing or preventing IBD, as well as identifying candidate genes which may be involved in the pathogenesis of IBD.

One aspect of the invention is therefore a method of inducing IBD-like symptoms in a mammal, wherein the mammal (i) is a non-human mammal not expressing a functional mdr1a gene product, and (ii) is subjected to elevated chlorine concentrations. Another aspect of the invention is a method of inducing IBD-like symptoms in a mammal, wherein the mammal is treated with an inhibitor of the mdr1a gene product or with a compound that inhibits the expression of the mdr1a gene product in the gut, and is subjected to elevated chlorine concentrations. Preferably the elevated chlorine concentrations are given by supply of chlorinated drinking water to the mammal. The chlorinated drinking water has a chlorine concentration of above about 1 ppm, preferably above about 3 ppm, most preferably about 5 ppm or above. Other ways of subjecting the mammals to elevated chlorine concentrations may include adding chlorine to the food supply, giving the animals chlorine tablets, or using chlorine-bleached bedding for the mammal, exposing the animals to bleach, etc. In a preferred aspect, the mammal is a transgenic rodent, preferably a transgenic mouse, not expressing a functional mdr1a gene. A preferred aspect of the invention is a method as described above wherein more than about 50%, preferably more than about 70%, more preferably more than about 80%, even more preferably more than about 90%, and most preferably about 100% of the animals show IBD-like symptoms.

The preferred mammal used in the method is a transgenic mdr1a^{-/-} knockout mouse, even more preferred transgenic mouse strain FVB. 129P₂₋Pgy3^{tm1} N7. However, any mammal not expressing the mdr1a gene product may be used. This may include a mammal with a naturally occurring mutation in both alleles of the mdr1a gene or its promoter. This may also include a wildtype mammal treated with a compound that inhibits the expression of the mdr1a gene in the mammal, e.g. a cAMP dependant protein kinase inhibitor, a sub-therapeutic dose of mitomycin C or an antisense oligonucleotide, ribozyme, siRNA (short interfering RNA) or other molecules interfering with the transcription or translation of the mdr1a gene or mRNA, specific for the mdr1a orthologue in the mammal of choice. Alternatively the mammal of choice expressing a functional mdr1a gene product may be used in the model provided the mdr1a gene product is effectively inhibited, e.g. by treatment with a compound that effectively inhibits the mdr1a gene product in the mammal, e.g. by treatment with a compound or compounds that have been identified as MDR inhibitors or by blocking antibodies specific for mdr1a. Various compounds and mechanisms to inhibit MDR have been reviewed recently (C. Avendano. & J. C. Menendez (2002) Curr. Med. Chem. 9, 159-193). As used herein, the term “effectively inhibited” refers to inhibition to less than about 60% of the normal activity, preferably less than about 80%, even more preferably less than about 90%.

Another aspect of the invention is a method of screening a candidate compound for its efficacy in ameliorating the symptoms of IBD, the method comprising the following steps:

(a) administering the candidate compound in a vehicle to a first non-human mammal not expressing a functional mdr1a gene product (or wherein the mdr1a gene product is effectively inhibited), subjected to elevated chlorine concentrations, preferably supplied as chlorinated drinking water;

(b) administering the vehicle without the candidate compound to a second non-human mammal not expressing a functional mdr1a gene product (or wherein the mdr1a gene product is effectively inhibited, or wherein the expression of the mdr1a gene product is inhibited), subjected to elevated chlorine concentrations, preferably supplied as chlorinated drinking water; and

(c) comparing the symptoms of IBD in the mammal(s) of (a) and (b),

wherein a decrease in symptoms of IBD in the mammal(s) of (a) as compared to the mammal(s) of (b) indicates efficacy of the compound.

A further aspect of the invention is a method of screening a candidate compound for its efficacy in preventing or delaying the development of IBD, the method comprising the following steps:

(a) administering the candidate compound in a vehicle to a first non-human mammal not expressing a functional mdr1a gene product (or wherein the mdr1a gene product is effectively inhibited, or wherein the expression of the mdr1a gene product is inhibited), before the onset of the disease in said mammal;

(b) administering the vehicle without the candidate compound to a second non-human mammal not expressing a functional mdr1a gene product (or wherein the mdr1a gene product is effectively inhibited, or wherein the expression of the mdr1a gene product is inhibited), before the onset of the disease in said mammal;

(c) subjecting the mammal(s) of (a) and (b) to elevated chlorine concentrations, preferably supplied as chlorinated drinking water; and

(d) comparing the onset of any symptoms of IBD in the animals, wherein a delay in or prevention of the onset of symptoms of IBD in the mammal(s) treated with the candidate compound in a vehicle compared to the mammal(s) treated with the vehicle without the candidate compound indicates efficacy of the compound.

Yet another aspect of the invention is a method of screening for genes that may be involved in the pathogenesis of IBD and therefore may be novel targets for the development of drugs for the treatment of IBD, comprising the following steps:

(a) subjecting a first non-human mammal not expressing a functional mdr1a gene product (or wherein the mdr1a gene product is effectively inhibited, or wherein the expression of the mdr1a gene product is inhibited) to elevated chlorine concentrations, preferably supplied as chlorinated drinking water;

(b) not subjecting a second non-human mammal not expressing a functional mdr1a gene product (or wherein the mdr1a gene product is effectively inhibited, or wherein the expression of the mdr1a gene product is inhibited) to elevated chlorine concentrations, preferably by only supplying unchlorinated drinking water and not otherwise supplying an elevated chlorine concentration;

(c) making RNA preparations from the intestine from both the mammals of (a) and of (b) after the desired time interval; and

(d) comparing the RNA samples,

wherein a RNA which shows a difference in these samples indicates a gene that may be implicated in the pathogenesis of IBD.

A further aspect of the invention is a method of screening for genes that may be involved in the pathogenesis of IBD comprising the following steps:

(a) subjecting a first non-human mammal not expressing a functional mdr1a gene product (or wherein the mdr1a gene product is effectively inhibited, or wherein the expression of the mdr1a gene product is inhibited) to elevated chlorine concentrations, preferably supplied as chlorinated drinking water;

(b) subjecting a second non-human mammal expressing a functional mdr1a gene product to elevated chlorine concentrations, preferably supplied as chlorinated drinking water;

(c) not subjecting a third non-human mammal not expressing a functional mdr1a gene product (or wherein the mdr1a gene product is effectively inhibited, or wherein the expression of the mdr1a gene product is inhibited) to elevated chlorine concentrations, preferably by supplying only unchlorinated drinking water and not otherwise supplying an elevated chlorine concentration;

(d) not subjecting a fourth non-human mammal expressing a functional mdr1a gene product to elevated chlorine concentrations, preferably by supplying only unchlorinated drinking water and not otherwise supplying an elevated chlorine concentration;

(e) making RNA preparations from the intestine from each of the mammals of (a) to (d) after the desired time interval; and

(f) comparing the RNA samples,

wherein a RNA which shows a difference between the samples of (a) and (c), but not a similar difference between the samples of (b) and (d), is a gene that may be implicated in the pathogenesis of IBD.

The comparison of the RNA samples mentioned above can be carried out by expression profiling, preferably by differential display PCR or subtractive hybridisation methods, even more preferably by microarray analysis.

The preferred mammal used in the methods described above is a transgenic mdr1a^{-/-} knockout mouse, even more preferred transgenic mouse strain FVB.129P2-Pgy3^{tm1} N7.

Another aspect of the invention is the identification of the human homologue of any candidate gene identified as described above. This can be carried out by using the mammalian candidate gene as a probe to screen a human cDNA library from suitable human gut tissue, followed by sequence analysis of the positive clones; it can also be done by searching databases such as Genbank (Nucl Acids Res. 30, 17-20 (2002)), available for example on the NCBI web site (http://www.ncbi.nlm.nih.gov/Genbank/GenbankOverview.html), with the sequence of the mammalian candidate gene. Other useful databases are the EMBL and DDBJ databases which are also part of the International Nucleotide Sequence Database collaboration. These methods and databases are well known to the skilled person. A preferred method for searching the databases would be Blast2 (Altschul, S. F. et al (1997) Nucl. Acids Res. 25, 3389-3402), but other methods well known to the skilled person, such as Fasta, can also be used. The human sequence with the highest similarity to the mammalian sequence used for the search will be the human homologue of the candidate gene in most cases. The skilled person will then be able to clone the full length cDNA of the human homologue of the candidate gene, e.g. by cDNA library screening or polymerase chain reaction (PCR) methods, and express it in suitable expression systems following methods as described in, for example, Sambrook, et al., eds. (1989) Molecular Cloning: A Laboratory Manual, Cold Spring Harbor Laboratory Press, New York, N.Y., USA or Ausubel, et al., eds. (1990) Current Protocols in Molecular Biology, John Wiley & Sons, Inc., or updated editions thereof, or other similar general texts.

A further aspect of the invention is a method of preparing a composition, which comprises

(a) identifying a compound that is capable of ameliorating the symptoms of IBD by the method comprising the following steps:

(i) administering the candidate compound in a vehicle to a first non-human mammal not expressing a functional mdr1a gene product (or wherein the mdr1a gene product is effectively inhibited), subjected to elevated chlorine concentrations, preferably by supply of chlorinated drinking water;

(ii) administering the vehicle without the candidate compound to a second transgenic non-human mammal not expressing a functional mdr1a gene product (or wherein the mdr1a gene product is effectively inhibited, or wherein the expression of the mdr1a gene product is inhibited), subjected to elevated chlorine concentrations, preferably by supply of chlorinated drinking water; and

(iii) comparing the symptoms of IBD in the mammal(s) of (i) and (ii),

wherein a decrease in symptoms of IBD in the mammal(s) of (i) as compared to the mammal(s) of (ii) indicates efficacy of the compound; and

(b) mixing said compound with a carrier.

Yet a further aspect of the invention is a method of preparing a composition which comprises

(a) identifying a compound that is capable of preventing or delaying the development of IBD by the method comprising the following steps:

(i) administering the candidate compound in a vehicle to a first non-human mammal not expressing a functional mdr1a gene product (or wherein the mdr1a gene product is effectively inhibited, or wherein the expression of the mdr1a gene product is inhibited), before the onset of the disease in said mammal;

(ii) administering the vehicle without the candidate compound to a second non-human mammal not expressing a functional mdr1a gene product (or wherein the mdr1a gene product is effectively inhibited, or wherein the expression of the mdr1a gene product is inhibited), before the onset of the disease in said mammal;

(iii) subjecting the mammal(s) of (i) and (ii) to elevated chlorine concentrations, preferably by supply of chlorinated drinking water; and

(iv) comparing the onset of any symptoms of IBD in the animals, wherein a delay in or prevention of the onset of symptoms of IBD in the mammal(s) treated with the candidate compound in a vehicle compared to the mammal(s) treated with the vehicle without the candidate compound indicates efficacy of the compound; and

(b) mixing said compound with a carrier.

### EXAMPLES

The examples are included for the purposes of illustration and are not intended to limit the scope of the invention. General methods referred to but not explicitly described herein are reported in the scientific literature and are well known to the skilled person.

### Example 1

### Mdr1a^{-/-} Mice as Animal Model for IBD

Materials and Methods

Animals

Forty male mdr1a^{-/-} mice (strain FVB.129P2-Pgy3^{tm1} N7) and six control male mice (mdr1a^{+/+}) from the same genetic background (FVB; strain FVB/NTac) (6-8 weeks old) were obtained from Taconic Farms (U.S.A). Mice were individually housed in solid bottom cages containing autoclaved Bee Kay bedding from Bantin & Kingman (U.K.) with the addition of Envirodri, a nesting material made from virgin paper, free from deodorising chemicals, to provide enrichment. Mice were fed on RAT & Mouse No 1 maintenance diet (RMI (E) SQC) (Special Diet services, Witham, Essex, U.K.) on arrival at Pfizer. For this study we used bottled water and Klortabs (5 ppm) (Arrowmight Biosciences, U.K.) to eliminate the possibility of water borne spreading of bacterial pathogens and to provide a water supply with constant levels of chlorine. Water was changed twice a week. This study was carried out in accordance with UK legislation and approved by Pfizer's internal ethical review process.

Study Design and Dosing

Prior to commencing the study, all mice (40 mdr1a^{-/-} and 6 mdr1a^{+/+}) acclimatised for 14 days. From a pilot study (Banner, K. H. et al (2001) Gastroenterology 120, A693) body weight and soft faeces score were found to be most predictive of GI inflammation and thus in the present study these clinical signs were monitored. A scoring system was devised with different weightings for different clinical signs (Table 1). The intention was that the score should reflect the severity of the colitis.

TABLE 1
Scoring sheet used for each mouse in the pilot study vs. modified
scoring sheet used in this study.

| Scoring system (pilot study) | | Scoring system (present study) | |
|---|---|---|---|
| Appearance | Score | | Score |
| Piloerection, hunched | 2 | | |
| Behaviour (choose one) | | | |
| Subdued, lethargic | 2 | | |
| Prostrated | 6 | | |
| Miscellaneous | | Miscellaneous | |
| Soiled anus | 1 | | |
| occult blood in faeces | 6 | | |
| soft “sticky” faeces | 6 | soft “sticky” faeces | 6 |
| Fresh blood (anus/faeces) | 6 | | |
| Body weight (choose one) | | Body weight (choose one) | |
| Absence of gain | 1 | Absence of gain | 1 |
| Body weight loss ≧ | 2 | Body weight loss ≧ | 2 |
| 0.5 g/day | | 0.5 g/day | |
| Body weight loss ≧ 20% | 6 | Body weight loss ≧ 20% | 6 |
| Total: | | Total: | |

| | |
|---|---|
| Judgement: | >11 clinical disease |
| 0-3 normal | close observation (3/day) |
| 4-10 clinical disease | |
| CONSIDER | |
| EUTHANASIA | |

Following the acclimatisation period, mdr1a^{-/-} mice with mild and moderate disease scores (mild disease (0-3) was defined by absence of gain or mild body weight loss (<0.5 g) and no other symptoms, whereas moderate disease (4-10) was defined by similar symptoms as a previous group but with soft faeces or greater loss in body weight (>20%) were randomised into 4 groups and dosed subcutaneously (s.c.) with vehicle (water, n=8) or dexamethasone (0.05 mg/kg, n=9; 0.3 mg/kg, n=8 or 2 mg/kg, n=8) daily for 7 days. The 6 mdr1a^{+/+} mice received vehicle (water). During the study period, one mouse from the group treated with 0.3 mg/kg dexamethasone was found dead and two were euthanised (one from the vehicle-treated group and one from the group treated with 0.05 mg/kg dexamethasone). At the end of the study all mice were euthanased. The colon and caecum from each mouse was removed and weighed. Each colon was then divided longitudinally so that one section could be processed for histological analysis and the other for cytokine measurement (see below).

Cytokine and Protein Measurement

Each individual colon sample was homogenised three times for 30 seconds at 4° C. in 3ml of ice-cold Krebs' solution (Sigma Chemical Co. U.K.). Samples were centrifuged for 15 min at 35,000 g (4° C.) and supernatants were removed. IL-8 and IFN-γ levels were then measured by ELISA kits (R&D systems, U.K.). The detection limits of these kits were 31.2-2000 pg/ml. Protein levels was assayed in supernatants using the method described by Bradford (Bradford, M. (1976) Analyt. Biochem. 72, 248-254), utilising Bradford Reagent (Sigma).

Histological Analysis

The animals were necropsied. The colon/rectum and caecum were weighed and colon/rectum, caecum, ileum, jejunum, duodenum, stomach, mesenteric nodes, thymus, kidney, spleen were sampled and fixed in 10% buffered formalin solution. The colon/rectum and ileum were prepared in a “Swiss roll” technique (Moolenbeek, C. & Ruitenberg E. J. (1981) Lab Anim 15(1):57-9) to evaluate their whole longitudinal section one slide. Tissues were processed routinely in an automatic tissue processor, embedded in paraffin, sectioned at 4-6 μm, stained with hematoxylin and eosin (HE), and examined histologically. The colitis was graded from 0 (no significant findings) to 4 (severe proliferative and ulcerative inflammation) according to the extent of the findings and severity of mucosal changes (Table 2) by a blinded observer. In addition, the mucosal height of the whole colon/rectum were assessed about every 100 μm with a standard interactive microcomputer-assisted image analysis system.

TABLE 2
Histological scoring system for colitis

| Score | |
|---|---|
| 0 | No significant findings |
| 1 | minimal multifocal mucosal changes involving the apex of the |
| | mucosal folds of the proximal colon |
| 2 | mild multifocal mucosal changes mainly localised within the |
| | mucosal folds of the proximal colon. Minimal involvement of the |
| | remaining areas |
| 3 | Marked mucosal changes involving diffusely the whole middle and |
| | distal colon and rectum. Mild multifocal changes in the proximal |
| | colon. |
| 4 | severe mucosal changes involving diffusely the whole middle |
| | and distal colon and rectum. Mild to moderate diffuse changes in |
| | the proximal colon. |

Colitis appeared to begin within the proximal region of the colon as this was the main region affected in scores 1 and 2. When the severity of changes increased (scores 3 and 4), lesions were more prominent in the middle and distal regions of the colon and in the rectum. The colitis was characterized histologically by variable infiltration of inflammatory cells within the lamina propna, mainly macrophages, lymphocytes and polynuclear neutrophils and mucosal thickening. Increase in crypt lengths, increased basophilia and number of mitoses of lining enterocytes, goblet cell loss and interstitial oedema were observed. While only a few erosions were seen in score 2, obliteration of normal architecture, surperficial to transmural ulcerations and crypt abscesses were seen from score 3.

Analysis of Pathogens

A number of pathogens such as Helicobacter hepaticus, Helicobacter bills, Helicobacter rodentium have been associated with the development of gastrointestinal inflammation in rodents. Three separate techniques were therefore used to confirm the presence or absence of all known murine pathogens in faecal and colon samples. Faecal samples were analysed using PCR, by two independent collaborators. Colon samples were analysed by electron microscopy and histology using Warthin Starry and Fite Faraco stains. Serological and bacteriological studies were also carried out according to FELASA recommendations (Report of FELASA working group (1996) Laboratory Animals 30, 193-208 & (1994) Laboratory Animals 28; 1-12).

Statistical Analysis

Data were analysed for the 29 mdr1a^{-/-} and 6 mdr1a^{+/+} mice, which completed the study. Differences between vehicle treated mdr1a^{-/-} and mdr1a^{+/+} mice were analysed using a two sample t-test (assuming equal variance for IFN-γ, growth rate in the acclimatisation period and final weight, and unequal variance for IL-8, colon weight, caecum weight, colon to body weight ratio, caecum to body weight ratio and growth rate during the study period). The effects of dexamethasone treatment were analysed using analysis of variance (ANOVA) allowing for culling group. Where treatment effects were found to be significant, then 95% confidence intervals were calculated.

Colitis severity score is an ordered categorical measure graded on a scale of 0-4. This type of data is generally analysed using a proportional odds model, however due to the small sample size this was not appropriate and ANOVA was used. This makes the assumption of having normally distributed data, which although we had categorical data, seemed to hold approximately true. All analysis was carried out using Genstat 5 Release 4.2 for Windows.

Results

Clinical Signs of Colitis

Growth Rate

There was no statistically significant difference in growth rate during the acclimatisation period between mdr1a^{-/-} and mdr1a^{+/+} mice (0.265 g/day vs. 0.294 g/day). However during the study period, there was a statistically significant difference between vehicle treated mdr1a^{-/-} and mdr1a^{+/+} mice. mdr1a+/+mice continued to gain weight whereas mdr1a^{-/-} mice lost weight (Table 3). In addition, at the end of the study, vehicle treated mdr1a^{-/-} mice were significantly lighter than mdr1a^{+/+} mice (Table 3). Dexamethasone treatment had no significant effect on either growth rate or final body weight at any of the doses used (Table 3).

TABLE 3
Growth Rate and body weight over study period
(allowing for culling group).

| | | Growth rate | Body | Clinical |
|---|---|---|---|---|
| | | (g/day) | weight (g) | score |
| | Treatment | (Mean ± | (Mean ± | (Mean ± |
| | (dosed s.c.) | s.e.m.) | s.e.m.) | s.e.m.) |
| mdr1a^{+/+} | vehicle | 0.149 ± 0.039 | 27.1 ± 0.55 | 0 |
| mdr1a^{-/-} | vehicle | −0.44 ± 0.099* | 22.8 ± 0.77* | 50 ± 8.9 |
| mdr1a^{-/-} | 0.05 mg/kg | −0.427 ± 0.099* | 22.2 ± 0.77* | 56 ± 8.9 |
| | dexamethasone | | | |
| mdr1a^{-/-} | 0.3 mg/kg | −0.260 ± 0.099* | 22.6 ± 0.77* | 37 ± 8.9 |
| | dexamethasone | | | |
| mdr1a^{-/-} | 2 mg/kg | −0.271 ± 0.093 | 22.7 ± 0.72* | 32 ± 8.3 |
| | dexamethasone | | | |

*Two sample t test: * P < 0.05 vs mdr1a^{+/+}

Clinical Scores

Dexamethasone had no effect on overall clinical scores of colitis (see Table 3 above).

Macroscopic Evidence of Colitis

Colon and Caecum Weights

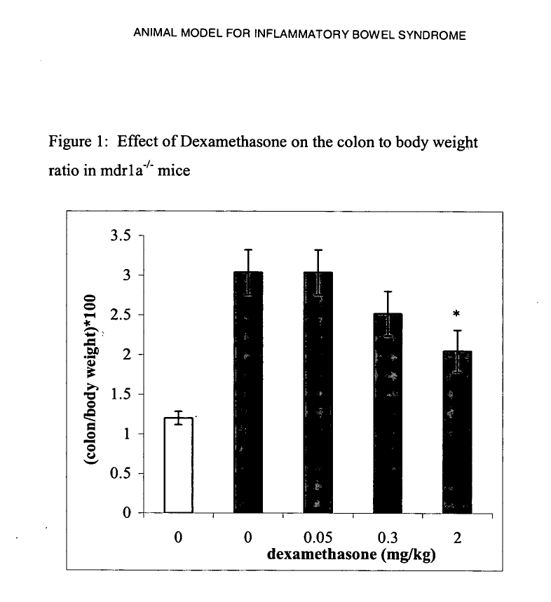

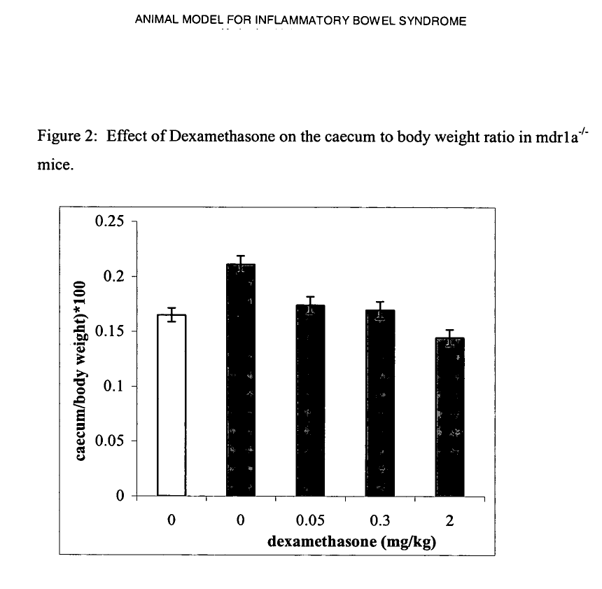

A significant increase in colon (691±69 vs. 325±26 mg) and caecum (211±12 vs. 165±6 mg) weight was observed in the vehicle treated mdr1a^{-/-} mice vs. mdr1a^{+/+} mice. Ratios of colon to body weight (FIG. 1, showing mean ±sem. Open bar represents mdr1a^{+/+} mice and filled bars represent mdr1a^{-/-} mice. * Statistically significant difference compared to 0 mg/kg mdr1a^{-/-} group (p<0.05).) and caecum to body weight (FIG. 2; showing mean ±sem. Open bar represents mdr1a^{+/+} mice and filled bars represent mdr1a^{-/-} mice. * Statistically significant difference compared to 0 mg/kg mdr1a^{-/-} group (p<0.05). ** Statistically significant difference compared to 0 mg/kg mdr1a^{-/-} group (p<0.01).) were also significantly higher in vehicle treated mdr1a^{-/-} mice vs. mdr1a^{+/+} mice, 3.0±0.31 vs. 1.2±0.08 and 0.9±0.05 vs. 0.6±0.02 respectively. Dexamethasone (2 mg/kg s.c. daily for 7 days) reduced absolute colon weight (456±48 vs. 691±52 mg; P<0.01) and colon to body weight ratio (2.1±0.26 vs. 3.0±0.28, P=0.017) vs. vehicle treated mdr1a^{-/-} mice, whilst lower doses of dexamethasone had no significant effect (FIG. 2). All doses of dexamethasone tested reduced absolute caecum weights (p<0.01) as well as caecum to body weight ratios (p<0.015) compared to vehicle treated mdr1a^{-/-} mice.

3. Microscopic Evidence of Colitis

Cytokine Levels

IL-8 (p=0.028) and IFN-γ (p<0.0l) levels were significantly higher in homogenised colons from vehicle treated mdr1a^{-/-} mice vs. mdr1a^{+/+} mice. Dexamethasone treatment however, had no effect on levels of either cytokine at any of the doses used (Table 4).

TABLE 4
Levels of IL-8 and IFN-γ in homogenised colon samples
from mdr1a^{+/+} and mdr1a^{-/-} mice (allowing for culling group)

| | | Mean ± s.e.m (pg/mg protein) | |
|---|---|---|---|
| | Treatment | IL-8 | IFN-γ |
| mdr1a^{+/+} | vehicle | 87 ± 10.6 | 26 ± 12.3 |
| mdr1a^{-/-} | vehicle | 208 ± 65.7 | 142 ± 32.6 |
| mdr1a^{-/-} | 0.05 mg/kg dexamethasone | 317 ± 65.7 | 177 ± 32.6 |
| mdr1a^{-/-} | 0.3 mg/kg dexamethasone | 264 ± 65.7 | 169 ± 32.6 |
| mdr1a^{-/-} | 2 mg/kg dexamethasone | 220 ± 61.5 | 100 ± 30.5 |

Histological Grading

Colons from mdr1a^{+/+} mice appeared normal. In contrast, colons from mdr1a^{-/-} mice were characterised by lesions similar to those observed in human IBD. Specifically, a severe spontaneous inflammation of the colon with a significant thickening of the mucosa was seen together with occasional crypt abscesses, increases in crypt length and ulcerations which extended through the mucosa to the muscle (which muscle layer). In addition, a dysregulated epithelial cell growth together with an inflammatory cell infiltrate into the lamina propria was observed. There was a decreasing trend in colitis severity score with increasing dose of Dexamethasone. There was not a statistically significant overall difference between the treatment means (p>0.10) but there was some evidence that the highest dose of Dexamethasone (2 mg/kg) significantly decreased the colitis severity score compared with that in the vehicle group. (Table 5).

TABLE 5
Effect of dexamethasone (0-2 mg/kg ) on colitis severity
scores graded histologically

| | | Mean ± | Difference | p- |
|---|---|---|---|---|
| | Treatment (dosed s.c.) | s.e.m. | from vehicle | value |
| mdr1a^{+/+} | vehicle | 0 | | |
| mdr1a^{-/-} | vehicle | 3.7 ± 0.42 | | |
| mdr1a^{-/-} | 0.05 mg/kg dexamethasone | 4.0 ± 0.42 | 0.3 | >0.05 |
| mdr1a^{-/-} | 0.3 mg/kg dexamethasone | 3.1 ± 0.42 | −0.6 | >0.05 |
| mdr1a^{-/-} | 2 mg/kg dexamethasone | 2.4 ± 0.39 | −1.3 | <0.05 |

Morphometry Assessment

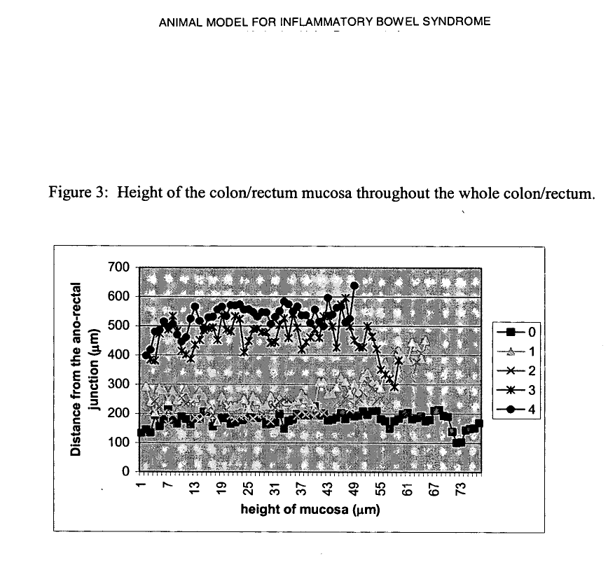

The morphometry assessement of the height of the mucosa was in agreement with the histological evaluation. When compared to Score 0, changes in mucosal height were close for scores 1 and 2 (minimal to mild, seen mainly in the proximal region of the colon) and for scores 3 and 4 (marked to severe, prominent in the middle and distal regions of the colon and in the rectum (FIG. 3; legend reflects colitis severity score from 0-4 (see Table 2 for details)). Also of note is the decrease in the length of the colon/rectum as the severity score of the colitis increases, a classical finding of colitis

Pathology

The absence of Helicobacters (H. Bilis, Hepaticus and spp) was confirmed by PCR, electron microscopy and histological staining. Absence of other know pathogens including citrobacter rodentium (bacteria associated with colonic hyperplasia) was confirmed by bacteriological and serological analysis. In summary, mice were free of viruses, bacteria and parasites known to be pathogenic as specified in FELASA recommendations (Report of FELASA working group (1996) Laboratory Animals 30, 193-208 & (1994) Laboratory Animals 28; 1-12).

### Example 2

### Controlled Study

16 mdr1a^{-/-} mice and 16 mdr1a^{+/+} mice of otherwise identical genetic background are acclimatised with unchlorinated drinking water. The animals are then divided into groups as shown in the Table below (Table 6):

TABLE 6
study design

| Group | Mouse | Drinking water |
|---|---|---|
| 1 | Mdr1a^{+/+} | unchlorinated |
| 2 | Mdr1a^{+/+} | 5 ppm chlorinated water |
| 3 | Mdr1a^{-/-} | unchlorinated |
| 4 | Mdr1a^{-/-} | 5 ppm chlorinated water |

The mice are then monitored for soft faeces and absence of body weight gain for up to 5 weeks.

Severe IBD-like symptoms are observed only in group 4.

### Example 3

### Compounds for the Treatment of IBD

40 mdr1a^{-/-} mice are acclimatised, and then given 5 ppm chlorinated drinking water for 2 days. They are then divided into 4 groups:

10 animals: given vehicle only

10 animals: given test compound at 0.05 mg/kg

10 animals: given test compound at 0.5 mg/kg

10 animals: given test compound at 5 mg/kg

Symptoms of IBD are monitored for 3-4 weeks, using one of the methods described in Example 1. A test compound is considered promising for the treatment of IBD if a significant, dose-related improvement is observed in the animals treated with the test compound as compared with the animals which are given vehicle only.

### Example 4

### Prevention of IBD

40 mdr1a^{-/-} mice are acclimatised for 2 days, during which they are given unchlorinated drinking water. They are then divided into 4 groups:

10 animals are given vehicle only

10 animals are given 0.05 mg/kg of test compound

10 animals are given 0.5 mg/kg of test compound

10 animals are given 5 mg/kg of test compound

The following day, the animals are supplied with chlorinated (5 ppm) drinking water. The animals are then monitored for the onset of IBD-like symptoms.

A test compound is considered promising for the prevention of IBD if a significant, dose-related decrease or delay in the onset of symptoms is observed in the animals treated with the test compound as compared with the animals which are given vehicle only.

### Example 5

### Comparison of Changes in Gene Expression in Colons from mdr1a^{-/-} vs Wild Type (FVB) Mice, and the Effect of Dexamethasone on These Changes

The aims of the study were to compare changes in gene expression in colons from FVB wildtype vs. mdr1a^{-/-} mice and to examine the effect of dexamethasone on these changes.

Male FVB wildtype and mdr1a^{-/-} mice (9-10 weeks old) were dosed with dexamethasone (2 mg/kg i.p.) or vehicle (water) daily for 7 days. Mice were culled and colons removed for histological assessment. RNA was purified from a single donor from each group using a modified Trizol and RNeasy protocol, yielding greater than 50 ug RNA/sample, with a A260/A280 purity greater than 1.9.

The procedure was performed according to Affymetrix manual: For each sample 10 μg of total RNA was reverse transcribed into double-stranded (ds) cDNA. Half of the dscDNA was In Vitro Transcribed (IVT) in the presence of biotinylated nucleotides. 25 μg of the IVT product was fragmented and about 15 μg was used in hybridisation cocktail to hybridise to Test 2 chip and then to U74A chips (see Affymetrix manual a.f.1,7307-1).

RNA quality and hybridisation signal were good in all samples. Mouse U74A data was normalised to 300. Total number of probe sets (˜genes) 12,639.

Analysis of the data showed that mdr1a^{-/-} mice showed clear evidence of increased inflammation as indicated by, for example, upregulation of inflammatory markers such as complement components, MHC molecules, leukocyte markers (indicating infiltration of leukocytes into the tissue), cytokines, chemokines, integrins, cyclooxygenase-2 and TNF family molecules and ligand, as well as showing evidence of tissue destruction as indicated by, for example, upregulation of metalloproteinase expression. Also shown was clear upregulation of known drug targets for IBD in the mdr1a^{-/-} mice as compared to the control animals, such as phospholipase A2 (about 12× upregulated), or inducible nitric oxide synthase (about 5× upregulated). This clearly indicates the suitability of the method for the identification of new drug targets that will aid in the identification of effective treatments for IBD.

Candidate drug targets are, for example, genes that are upregulated in mdr1a^{-/-} mice and that are brought down to near normal levels by the dexamethasone treatment.

Candidate biomarkers, useful in diagnosis and assessment of disease status, progression, and success of therapy, can also be identified by such an experiment; for example, we found known markers for IBD such as interleukin-1β (61 fold upregulated), matrix metalloproteinase 3 (MMP-3; 29 fold upregulated) or granzyme B (6 fold upregulated), upregulated in the mdr1a^{-/-} mice as compared to the control animals. Other genes showing the same pattern of regulation in this experiment are therefore also candidate biomarkers or diagnostic tools for IBD.

### Example 6

### Time Course of Changes in Gene Expression in mdr1a^{-/-} Mice After Switch to Chlorinated Drinking Water as Compared to Wildtype Mice

To identify genes involved in the pathogenesis of IBD, changes in gene expression in the colon in mdr1a^{-/-} mice as compared to the FVB wildtype mice are studied over time after switching the mice to chlorinated drinking water.

Male mice (9-10 weeks old) (same number of wildtype and mdr1a^{-/-}) are acclimatised, receiving unchlorinated drinking water, for 2 days. The water is then changed to chlorinated drinking water (5 ppm), and mice from each group in parallel are culled and colons removed for histological assessment and RNA isolation just before and at various time points after they have started drinking the chlorinated water. Suitable time points would be 2 hours, 4 hours, 6 hours, 8 hours, 12 hours, 24 hours, 48 hours, 72 hours.

The expression profiling is carried out as described in the previous Example.

Genes that are candidates for being involved in the pathogenesis of IBD are those that change in expression in mdr1a^{-/-} mice but not (or not to the same extent) in wildtype mice. Especially interesting are genes that change early after switching the water supply, as these may be genes involved in the onset of the disease, and inhibiting those may prevent the onset of the disease.

Genes that are candidates for being useful biomarkers for IBD, either for assessing disease progression or success of treatment can also be identified by this experiment. For example, such genes may encode proteins that are found in serum, and useful biomarkers may be upregulated or downregulated in the mdr1a^{-/-} mice as compared to the wildtype mice.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. Accordingly, the invention is limited only by the following claims.

1. A method of inducing IBD-like symptoms in a mammal, wherein the mammal (i) is a non-human mammal not expressing a functional mdr1a gene product, and (ii) is subjected to elevated chlorine concentrations.

2. A method of inducing IBD-like symptoms in a mammal, wherein the mammal is treated with an inhibitor of the mdr1a gene product and is subjected to elevated chlorine concentrations.

3. The method of claim 1 or claim 2, wherein the elevated chlorine concentrations are administered by supply of chlorinated drinking water to the mammal.

4. The method of claim 3, wherein the concentration of chlorine in the drinking water is above about 1 ppm.

5. The method of claim 1, wherein the mammal is a transgenic mdr1a^{-/-} knockout mouse.

6. The method of claims 1 or 2, wherein IBD-like symptoms occur in about 100% of the mammals.

7. A method of screening a candidate compound for its efficacy in ameliorating the symptoms of IBD, the method comprising the following steps:
(a) administering the candidate compound in a vehicle to a first non-human mammal not expressing a functional mdr1a gene product or wherein the mdr1a gene product or its expression is effectively inhibited, subjected to elevated chlorine concentrations;
(b) administering the vehicle without the candidate compound to a second non-human mammal not expressing a functional mdr1a gene product or wherein the mdr1a gene product or its expression is effectively inhibited, subjected to elevated chlorine concentrations; and
(c) comparing the symptoms of IBD in the mammal(s) of steps (a) and (b),
wherein a decrease in symptoms of IBD in the mammal(s) of step (a) as compared to the mammal(s) of step (b) indicates efficacy of the compound.

8. A method of screening a candidate compound for its efficacy in preventing or delaying the development of IBD, the method comprising the following steps:
(a) administering the candidate compound in a vehicle to a first non-human mammal not expressing a functional mdr1a gene product or wherein the mdr1a gene product or its expression is effectively inhibited, before the onset of the disease in said mammal;
(b) administering the vehicle without the candidate compound to a second non-human mammal not expressing a functional mdr1a gene product or wherein the mdr1a gene product or its expression is effectively inhibited, before the onset of the disease in said mammal;
(c) subjecting the mammal(s) of step (a) and step (b) to elevated chlorine concentrations; and
(d) comparing the onset of any symptoms of IBD in the animals,
wherein a delay in or prevention of the onset of symptoms of IBD in the mammal(s) treated with the candidate compound in a vehicle compared to the mammal(s) treated with the vehicle without the candidate compound indicates efficacy of the compound.

9. A method of screening for genes that may be involved in the pathogenesis of IBD comprising the following steps:
(a) subjecting a first non-human mammal not expressing a functional mdr1a gene product or wherein the mdr1a gene product or its expression is effectively inhibited to elevated chlorine concentrations;
(b) not subjecting a second non-human mammal not expressing a functional mdr1a gene product or wherein the mdr1a gene product or its expression is effectively inhibited to elevated chlorine concentrations;
(c) making RNA preparations from the intestine from both the mammals of step (a) and of step (b) after the desired time interval; and
(d) comparing the RNA samples,
wherein a RNA which shows a difference in these samples indicates a gene that may be implicated in the pathogenesis of IBD.

10. A method of screening for genes that may be involved in the pathogenesis of IBD comprising the following steps:
(a) subjecting a first non-human mammal not expressing a functional mdr1a gene product or wherein the mdr1a gene product or its expression is effectively inhibited to elevated chlorine concentrations;
(b) subjecting a second non-human mammal expressing a functional mdr1a gene product to elevated chlorine concentrations;
(c) not subjecting a third non-human mammal not expressing a functional mdr1a gene product or wherein the mdr1a gene product or its expression is effectively inhibited to elevated chlorine concentrations;
(d) not subjecting a fourth non-human mammal expressing a functional mdr1a gene product to elevated chlorine concentrations;
(e) making RNA preparations from the intestine from each of the mammals of steps (a) to (d) after the desired time interval; and
(f) comparing the RNA samples,
wherein a RNA which shows a difference between the samples of step (a) and step (c), but not a similar difference between the samples of step (b) and step (d), is a gene that may be implicated in the pathogenesis of IBD.

11. The method of claim 9 or 10, wherein the RNA samples are compared by expression profiling.

12. The method of claim 11, wherein the expression profiling is performed by microarray analysis.

13. The method of one of claims 9 or 10, further comprising the step of identifying the human homologue of the identified gene.

14. The method of one of claims 7 to 10, wherein the elevated chlorine concentrations are administered by supply of chlorinated drinking water to the mammal and wherein the concentration of chlorine in the drinking water is above about 1 ppm.

15. The method of claims 9 or 10, wherein the step of not subjecting the mammal to elevated chlorine concentrations is carried out by supplying only unchlorinated drinking water to the mammal and not otherwise supplying an elevated chlorine concentration to the mammal.
